(19) United States
(12) Patent Application Publication
(10) Pub. No.: US 2006/0115695 A1
(43) Pub. Date: Jun. 1, 2006

(54) Bisphenol compound and aromatic polyaryl ether

(75) Inventors: Tetsuji Hirano, Yamaguchi (JP); Tatsuya Arai, Yamaguchi (JP); Masayuki Kinouchi, Yamaguchi (JP)

Correspondence Address: YOUNG & THOMPSON, 745 SOUTH 23RD STREET, 2ND FLOOR, ARLINGTON, VA, 22202, US

(73) Assignee: UBE INDUSTRIES, LTD., Ube-Shi (JP)

(21) Appl. No.: 10/540,984

(22) PCT Filed: Dec. 25, 2003
(86) PCT No.: PCT/JP03/16756

§ 371 Date: Jun. 27, 2005

(30) Foreign Application Priority Data
Dec. 26, 2002 (JP) 2002-376593
Feb. 17, 2003 (JP) 2003-37791

(51) Int. Cl. H01M 8/18; C07C 309/29

(52) U.S. Cl. 429/20; 525/87; 562/42

(57) ABSTRACT

A novel bisphenol compound comprising an alkylsulfonic acid and/or an alkali metal salt thereof is disclosed. The novel bisphenol compound is preferably represented by the chemical formula shown below. A novel aromatic polyaryl ether synthesized using the bisphenol compound and carrying an alkylsulfonic acid and/or an alkali metal salt thereof in the side chain is also disclosed. (wherein R⁵ represents CH₃ or a phenyl group; p and q are independently an integer from 1 to 12; and X represents a hydrogen atom or an alkali metal.)

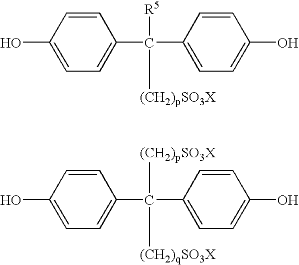

### TECHNICAL FIELD

This invention relates to a novel bisphenol compound having an alkylsulfonic acid and/or an alkali metal salt thereof and to an aromatic polyaryl ether. The novel bisphenol compound of the present invention is useful as a starting material of polyelectrolytes comprising aromatic polyether sulfones, aromatic polyether ketones, aromatic polyesters, polycarbonates, epoxy resins, phenol resins, and so forth. The novel aromatic polyaryl ether of the present invention is useful as a molding material of polyelectrolytes or polyelectrolyte membranes for use in fuel cells, secondary batteries, capacitors, ion exchange resins, ion exchange membranes, separation membranes.

### BACKGROUND OF INVENTION

Bisphenol compounds are used as a starting material for aromatic polyether sulfones, aromatic polyether ketones, aromatic polyesters, polycarbonates, epoxy resins, and phenol resins, and the like.

Aromatic polyaryl ethers such as aromatic polyether sulfones and aromatic polyether ketones become polyelectrolytes upon introducing a sulfonic acid group, which are used as ion exchange resins, ion exchange membranes, polyelectrolyte membranes for fuel cells, and the like. Most of such polyelectrolytes have a sulfonic acid group introduced directly to the aromatic rings. However, the problem of a sulfonic acid group directly bonded to an aromatic ring is that it releases easily in the presence of water under an acidic condition as described, e.g., in Hirotada Iida, Yuki gosei kagaku, Baifukan, Tokyo, p. 139 (1975).

Alkylsulfonated polyether sulfones or polyether ketones are disclosed, e.g., in A. Higuchi, et al., J. Appl. Polym. Sci., vol. 36, 1753 (1988) and JP-A-2002-110174. These polyaryl ethers are obtained by alkylsulfonating corresponding polymers (polymer reaction). However, polymer reaction involves disadvantages such as tendencies to non-uniformity of reaction and difficulties in reaction control and molecular design. It has therefore been demanded to develop a dihydric phenol compound having an alkylsulfonic group that can serve as a starting material of the above-mentioned polymers and an alkylsulfonated aromatic polyaryl ether synthesized from the phenol compound.

### DISCLOSURE OF THE INVENTION

A first object of the present invention is to provide a novel bisphenol compound having an alkylsulfonic acid and/or an alkali metal salt thereof, which compound is useful as a starting material of polyelectrolytes comprising aromatic polyether sulfones, aromatic polyether ketones, aromatic polyesters, polycarbonates, epoxy resins, phenol resins, etc.

A second object of the invention is to provide a novel aromatic polyaryl ether, such as an aromatic polyether sulfone or an aromatic polyether ketone, which carries an alkylsulfonic acid group and/or an alkali metal salt thereof in the side chain thereof.

The present inventors have conducted extensive studies with the first object in mind and, as a result, synthesized a novel bisphenol compound having an alkylsulfonic acid and/or an alkali metal salt thereof and thus reached the present invention.

The bisphenol compound according to the present invention is represented by chemical formula (1): 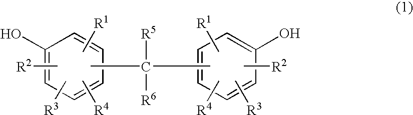
wherein R¹, R², R³, and R⁴ each independently represent a hydrogen atom or an alkyl group having 1 to 3 carbon atoms; and R⁵ and R⁶ each independently represent a hydrogen atom, an alkyl group having 1 to 6 carbon atoms, an aromatic group or a structure represented by chemical formula (2):
—R⁷(CH₂)ₙSO₃X (2)
wherein R⁷ represents nothing or an aromatic group; X represents a hydrogen atom or an alkali metal; and n represents an integer of from 1 to 12, provided that at least one of R⁵ and R⁶ represents the structure of chemical formula (2).

As a result of further studies, the present inventors have accomplished the second object by synthesizing an aromatic polyaryl ether starting with the above-described bisphenol compound of the present invention.

That is, the aromatic polyaryl ether of the invention is characterized by having a structural unit represented by chemical formula (4): 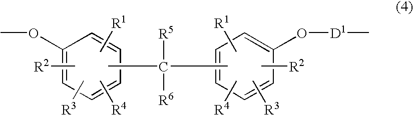
wherein R¹, R², R³, and R⁴ each independently represent a hydrogen atom or an alkyl group having 1 to 3 carbon atoms; R⁵ and R⁶ each independently represent a hydrogen atom, an alkyl group having 1 to 6 carbon atoms, an aromatic group or a structure represented by chemical formula (5):
—R⁷(CH₂)ₙSO₃X (5)
wherein R¹ represents nothing or an aromatic group; X represents a hydrogen atom or an alkali metal; and n represents an integer of from 1 to 12,
provided that at least one of R⁵ and R⁶ represents the structure of chemical formula (5); and D¹ represents a structure represented by chemical formulae (6) or (7): 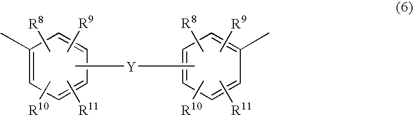
wherein R⁸, R⁹, R¹⁰, and R¹¹ each independently represent a hydrogen atom, a halogen atom, an alkyl group having 1 to 3 carbon atoms or a nitro group; and Y represents —S(═O)₂ or —C(═O)—. 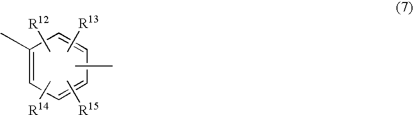
wherein R¹², R¹³, R¹⁴, and R¹⁵ each independently represent a hydrogen atom, a halogen atom, an alkyl group having 1 to 3 carbon atoms, a nitro group or a cyano group, provided that at least one of them is a nitro group or a cyano group.

### BEST MODE FOR CARRYING OUT THE INVENTION

The bisphenol compound represented by chemical formula (1) will be described first.

In chemical formula (1), R¹ through R⁴ are each preferably a hydrogen atom or CH₃, still preferably a hydrogen atom.

R⁵ and R⁶ each preferably represent CH₃, a phenyl group or the structure of chemical formula (2), provided that at least one of them is the structure represented by chemical formula (2). In chemical formula (2), R⁷ is preferably nothing, and X is preferably hydrogen or an alkali metal, e.g., Na or K.

The bisphenol compound of the invention is preferably a compound represented by chemical formula (3): 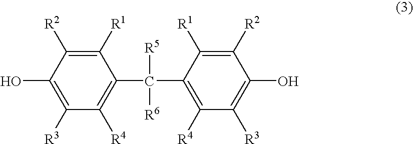
wherein R¹ through R⁶ are the same as R¹ through R⁶ in chemical formula (1).

The bisphenol compound of the invention is particularly preferably represented by chemical formulae (9) or (10): 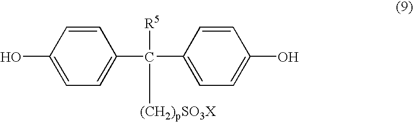
wherein R⁵ represents CH₃ or a phenyl group; p represents an integer of from 1 to 12; and X represents hydrogen or an alkali metal such as Na or K. 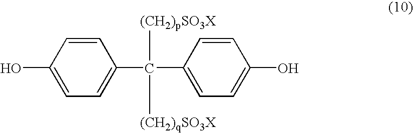
wherein p and q each independently represent an integer of 1 to 12; and X represents hydrogen or an alkali metal such as Na or K.

The process of preparing the bisphenol compound of the invention will then be described.

The bisphenol compound with an alkylsulfonic acid group and/or an alkali metal salt thereof according to the present invention is synthesized by sulfonating a halogenated bisphenol compound at the halogen atom, the halogenated bisphenol compound being represented by chemical formula (11): 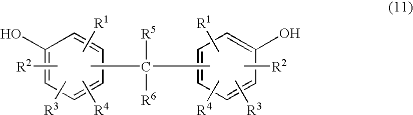
wherein R¹ to R⁴ each independently represent a hydrogen atom or an alkyl group having 1 to 3 carbon atoms; and R⁵ and R⁶ each independently represent a hydrogen atom, an alkyl group having 1 to 6 carbon atoms, an aromatic group or a structure represented by chemical formula (12):
—R⁷(CH₂)ₙZ (12)
wherein R⁷ represents nothing or an aromatic group; Z represents a halogen atom; and n represents an integer of 1 to 12,
provided that at least one of R⁵ and R⁶ represents the structure of chemical formula (12).

The halogenated bisphenol compound represented by chemical formula (11) is prepared using processes of synthesizing known bisphenols typified by bisphenol A. For example, it is synthesized from a halogenated ketone or aldehyde compound and a monohydric phenol in the presence of an acid catalyst and, if desired, a cocatalyst as taught in U.S. Pat. No. 6,353,079.

The halogenated ketone or aldehyde compound used in the synthesize of the halogenated bisphenol compound of chemical formula (11) is a compound having one ketone or aldehyde group per molecule and also having at least one halogen group bonded to an alkyl group. Specific examples thereof include chloroacetaldehyde, chloroacetone, 4-chloro-2-butanone, 3-chloro-2-butanone, 1-chloro-3-pentanone, 5-chloro-2-pentanone, 1-chloro-5-hexanone, 1-chloro-3,3-dimethyl-2-butanone, 2-chloroacetophenone, 4-chlorobutyrophenone, 2-chloro-4′-fluoroacetophenone, 4-chloro-4′-fluorobutyrophenone, 3-chloro-4′-fluoropropiophenone, 4-chloro-4′-methoxybutyrophenone, 3-chloropropiophenone, 2-bromoacetophenone, 2-bromo-4′-chloroacetophenone, 4′-bromo-3-chloropropiophenone, 4′-bromo-4-chlorobutyrophenone, 2-bromo-2′,4′-dimethoxyacetophenone, 2-bromo-2′,5′-dimethoxyacetophenone, 2-bromo-4′-fluoroacetophenone, 2-bromoisobutyrophenone, 2-bromo-2′-methoxyacetophenone, 2-bromo-3′-methoxyacetophenone, 2-bromo-4′-methoxyacetophenone, 2-bromo-4′-methylacetophenone, 4-(bromomethyl)benzophenone, 2-bromo-4′-phenylacetophenone, 1-bromopinacolone, 2-bromopropiophenone, 4-(chloroacetyl)catechol, 2-chloropropiophenone, 3-chloropropiophenone, 1,3-dichloro-2-propanone, 1,5-dichloro-3-pentanone, 1,7-dichloro-4-heptanone, 1,8-dichloro-4-octanone, 1,9-dichloro-5-nonanone, 1,3-dibromo-2-propanone, 1,5-dibromo-3-pentanone, 1,7-dibromo-4-heptanone, 1,8-dibromo-4-octanone, and 1,9-dibromo-5-nonanone).

The monohydric phenol compound used to synthesize the halogenated bisphenol compound of chemical formula (11) includes phenol, o-cresol, m-cresol, p-cresol, 2-ethylphenol, 3-ethylphenol, 4-ethylphenol, 2-propylphenol, 4-propylphenol, 2,3-dimethylphenol, 2,4-dimethylphenol, 2,5-dimethylphenol, 2,6-dimethylphenol, 3,4-dimethylphenol, 3,5-dimethylphenol, 2,3,5-trimethylphenol, and 2,3,6-trimethylphenol. Particularly preferred are phenol, o-cresol, m-cresol, 2,3-dimethylphenol, 2,5-dimethylphenol, and 2,6-dimethylphenol in view of their reactivity and availability and also with consideration given to the reactivity of the bisphenol compound of the invention in the production of the aromatic polyaryl ether of the invention.

In the reaction between the halogenated ketone or aldehyde compound and the monohydric phenol compound, there is no particular limitation to the ratio of the two reactants. It is desirable to minimize the amount of unreacted halogenated ketone or aldehyde compound from the viewpoint of economy and the ease of purifying the produced bisphenol compound. For this, it is advantageous to use the monohydric phenol compound in excess of stoichiometry. Specifically, the monohydric phenol compound is used in an amount of 3 to 200 mol, preferably 5 to 150 mol, per mole of the halogenated ketone or aldehyde compound.

The reaction temperature ranges usually from 30° to 150° C., preferably from 35° to 110° C. At reaction temperatures lower than 30° C., the reaction rate is low, and the reaction system can sometimes solidify. At temperatures higher than 150° C., on the other hand, it is difficult to control the reaction, which can result in an increased amount of a by-product. Usually, a solvent is unnecessary because the monohydric phenol compound functions as a solvent.

The acid catalyst used in the synthesis of the halogenated bisphenol compound represented by chemical formula (11) includes hydrochloric acid, sulfuric acid, alkylsulfonic acids, aromatic sulfonic acids, and sulfonic acid type ion exchange resins, such as sulfonated styrene-divinylbenzene copolymers, sulfonated crosslinked styrene polymers, phenol formaldehyde-sulfonic acid resins, benzene formaldehyde-sulfonic acid resins, and perflurocarbonsulfonic acid resins. The acid catalyst is used in an amount usually of from 0.05 to 30 mol %, preferably of from 0.1 to 25 mol %, based on the halogenated ketone or aldehyde compound.

The cocatalyst, which is used when needed in the synthesis of the bisphenol compound represented by chemical formula (11), includes mercaptans, i.e., compounds having an SH group in the molecule, such as alkyl mercaptans, alkyl mercaptans having one or more substituents selected from a carboxyl group, an amino group, a hydroxyl group, etc. (e.g., mercaptocarboxylic acids, aminoalkanethiols, and mercaptoalcohols). Examples of the mercaptans include alkyl mercaptans such as methyl mercaptan, ethyl mercaptan, n-butyl mercaptan, and n-octyl mercaptan; thiocarboxylic acids such as thioglycolic acid and β-mercaptopropionic acid; aminoalkanethiols such as 2-aminoethanethiol and 2,2-dimethylthiazolidine; and mercaptoalcohols such as mercaptoethanol. These mercaptans can be used either individually or as a combination of two or more thereof. The mercaptan is used in an amount usually of 0.1 to 30 mol %, preferably of 0.15 to 25 mol %, based on the halogenated ketone or aldehyde compound. Mercaptosulfonic acids such as 3-mercapto-1-propanesulfonic acid, can be used as an acid catalyst also functioning as a cocatalyst.

If desired, the synthesized halogenated bisphenol compound is purified by solvent washing, extraction, column fractionation, and the like.

Sulfonation of the halogenated bisphenol compound of chemical formula (11) at the halogen group is achieved by allowing the halogenated bisphenol compound and sodium sulfite or potassium sulfite to react in a solvent under reflux for 0.5 to 72 hours. The sodium sulfite or potassium sulfite is used in an amount of 1 to 10 mol, preferably 1.1 to 5 mol, per mole of the halogen group of the halogenated bisphenol compound. The solvent is preferably water or a water/acetone mixed solvent. Acetone is used at a ratio of 0.5% to 60%, preferably 1% to 50%, by weight in the mixed solvent.

If necessary, the resulting alkylsulfonated bisphenol compound alkyl metal salt can be purified by solvent washing, extraction, column fractionation, and the like. Treatment of the metal salt with the aforementioned acid catalyst or an aqueous solution thereof results in removal of the alkali metal to give a sulfonic acid. The resulting sulfonic acid may be converted to another alkali metal.

The aromatic polyaryl ether according to the present invention, which has the structural unit represented by chemical formula (4), will now be described.

The aromatic polyaryl ether of the invention is preferably one in which the structural unit represented by chemical formula (4) is represented by chemical formula (8): 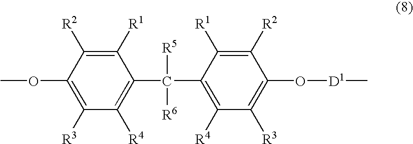
wherein R¹, R², R³, R⁴, R⁵, R⁶, and D¹ are as defined in chemical formula (4).

More specifically, the aromatic polyaryl ether of the invention preferably includes those represented by the following chemical formulae. 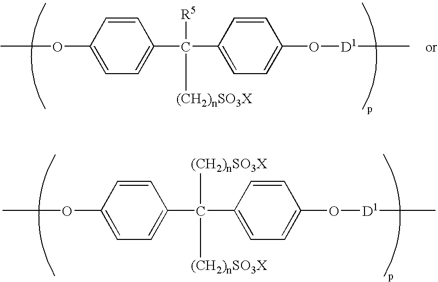
wherein R⁵ represents a hydrogen atom, an alkyl group having 1 to 6 carbon atoms or an aromatic group; D¹ represents a structure represented by: 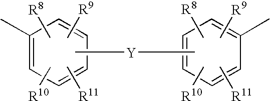
wherein R⁸, R⁹, R¹⁰, and R¹¹ each independently represent a hydrogen atom, a halogen atom, an alkyl group having 1 to 3 carbon atoms or a nitro group; and Y represents —S(═O)₂— or —C(═O)—,
or a structure represented by: 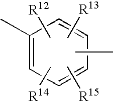
wherein R¹², R¹³, R¹⁴, and R¹⁵ each independently represent a hydrogen atom, a halogen atom, an alkyl group having 1 to 3 carbon atoms, a nitro group or a cyano group, provided that at least one of them is a nitro group or a cyano group;
m and n each independently represent an integer of 1 to 12; p represents an integer of 5 to 2500; and X represents a hydrogen atom or an alkali metal.

Still more specifically, particularly preferred examples of the aromatic polyaryl ether polymers of the invention include those represented by the chemical formulae shown below: 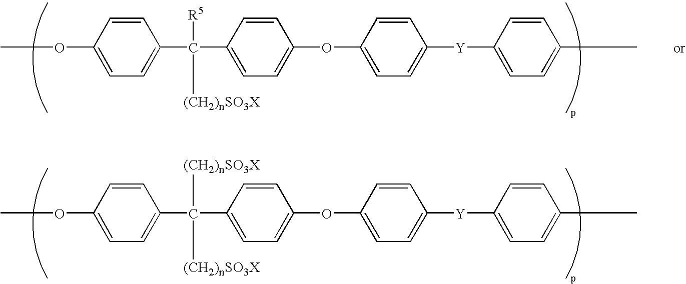
wherein R⁵ represents a hydrogen atom, an alkyl group having 1 to 6 carbon atoms or an aromatic group; Y represents —S(═O)₂— or —C(═O)—; m and n each independently represent an integer of 1 to 12; p represents an integer of 5 to 2500; and X represents a hydrogen atom or an alkali metal.

The aromatic polyaryl ether according to the invention having an alkylsulfonic acid and/or an alkali metal salt thereof is obtained using the alkylsulfonated bisphenol compound alkyl metal salt (i.e., the bisphenol compound of the invention), which is a dihydric phenol, as a starting compound. It is synthesized by, for example, nucleophilic substitution reaction between a dialkali metal salt of a dihydric phenol and an aromatic dihalide having an electron attracting group as described in Ueda Mitsuru, Shin-kobunshi jikkengaku 3 kobunshino gosei han-no (2), Kyoritsu Shuppan Co., Ltd., Tokyo, p. 10-24 (1996).

The aromatic dihalide that can be used to synthesize the aromatic polyaryl ether of the invention includes those having a sulfone group, such as bis(4-chlorophenyl) sulfone, bis(4-fluorophenyl) sulfone, bis(4-bromophenyl) sulfone, bis(4-iodophenyl) sulfone, bis(2-chlorophenyl) sulfone, bis(2-fluorophenyl) sulfone, bis(2-methyl-4-chlorophenyl) sulfone, bis(2-methyl-4-fluorophenyl) sulfone, bis(3,5-dimethyl-4-chlorophenyl) sulfone, and bis(3,5-dimethyl-4-fluorophenyl) sulfone; those having a ketone group, such as 4,4′-difluorobenzophenone, 2,4′-difluorobenzophenone, and 4,4′-dichlorobenzophenone; and those having a nitrile group, such as 2,6-difluorobenzonitrile. These dihalides can be used either individually or as a combination of two or more thereof. Preferred of them are bis(4-chlorophenyl) sulfone, bis(4-fluorophenyl) sulfone, 4,4′-difluorobenzophenone, and 4,4′-dichlorobenzophenone in view of their availability and reactivity.

The reaction between the alkylsulfonated bisphenol compound alkyl metal salt (the bisphenol compound of the invention) as a dihydric phenol dialkali metal salt and the aromatic dihalide is carried out using a polar solvent, such as dimethyl sulfoxide, sulfolane, N-methyl-2-pyrrolidone, 1,3-dimethyl-2-imidazolidinone, N,N-dimethylformamide, N,N-dimethylacetamide, and diphenyl sulfone. The reaction temperature preferably ranges from 140° to 320° C. The reaction time is preferably 0.5 to 100 hours.

The aromatic polyaryl ether having an alkylsulfonic acid and/or an alkali metal salt thereof according to the present invention also includes copolymers having a structural unit represented by chemical formula (13) or (14) shown below which are prepared by additionally using a dihydric phenol other than the alkylsulfonated bisphenol compound alkyl metal salt. 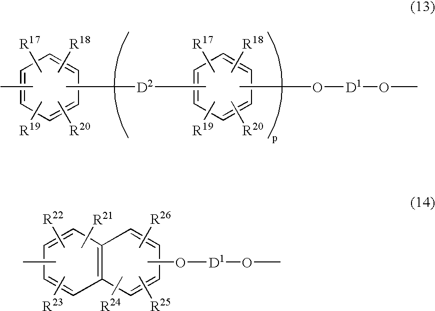
wherein R¹⁷, R¹⁸, R¹⁹, R²⁰, R²¹, R²², R²³, R²⁴, R²⁵, and R²⁶ each independently represent a hydrogen atom or an alkyl group having 1 to 3 carbon atoms; D² represents nothing, —O—, —CH₂—, —S(═O)₂—, —C(═O)—, —S— or —C(CH₃)₂—; p represents an integer of from 0 to 3; and D¹ represents a structure represented by 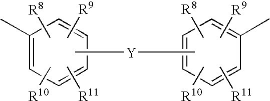
wherein R⁸, R⁹, R¹⁰, and R¹¹ each independently represent a hydrogen atom, a halogen atom, an alkyl group having 1 to 3 carbon atoms or a nitro group; and Y represents —S(═O)₂— or —C(═O)—,
or a structure represented by 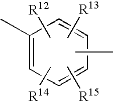
wherein R¹², R¹³, R¹⁴, and R¹⁵ each independently represent a hydrogen atom, a halogen atom, an alkyl group having 1 to 3 carbon atoms, a nitro group or a cyano group, provided that at least one of them represents a nitro group or a cyano group.

The copolymer having the structural unit represented by chemical formula (13) or (14) may be either a random copolymer or a block copolymer. The proportion of the structural unit having no alkylsulfonic acid alkali metal salt (i.e., the structural unit of chemical formula (13) or (14)) in the copolymer is not more than 95% by weight, preferably 90% by weight or less, more preferably 85% by weight or less. With the proportion of the structural unit with no alkylsulfonic acid alkali metal salt exceeding 95% by weight, the copolymer hardly displays the characteristics.

Examples of the dihydric phenol compound other than the alkylsulfonated bisphenol compound alkyl metal salt, which can be used in the synthesis of the aromatic polyaryl ether copolymer, include hydroquinone, resorcinol, 1,5-dihydroxynaphthalene, 1,6-dihydroxynaphthalene, 1,7-dihydroxynaphthalene, 2,7-dihydroxynaphthalene, 4,4′-biphenol, 2,2′-biphenol, bis(4-hydroxyphenyl) ether, bis(2-hydroxyphenyl) ether, 2,2-bis(4-hydroxyphenyl)propane, 2,2-bis(3-methyl-4-hydroxyphenyl)propane, 2,2-bis(3,5-dimethyl-4-hydroxyphenyl)propane, bis(4-hydroxyphenyl)methane, bis(4-hydroxyphenyl) sulfone, bis(4-hydroxyphenyl) sulfide, bis(4-hydroxyphenyl) ketone, 2,2-bis(3,5-dimethyl-4-hydroxyphenyl)hexafluoropropane, and 9,9-bis(4-hydroxyphenyl)fluorene. Preferred of them are hydro quinone, 4,4′-biphenol, bis(4-hydroxyphenyl) ether, 2,2-bis(4-hydroxyphenyl)propane, bis(4-hydroxyphenyl)methane, bis(4-hydroxyphenyl) sulfone, 1,5-dihydroxynaphthalene, and 2,7-dihydroxynaphthalene because of their availability and reactivity.

The aromatic polyaryl ether having an alkylsulfonic acid and/or an alkali metal salt thereof according to the invention preferably has a degree of polymerization of 5 to 2500, still preferably 10 to 2000. With a polymerization degree smaller than 5, the polymer hardly manifest the characteristics. With a polymerization degree greater than 2500, the polymer is difficult to form into a membrane.

The alkali metal alkylsulfonate group of the aromatic polyaryl ether of the present invention can easily be converted to an alkylsulfonic acid by treating with an aqueous solution of hydrochloric acid, sulfuric acid, etc. The conversion may be conducted either after the synthesis or after molding into a membrane, a sheet, a fiber (inclusive of a hollow fiber) or other molded articles.

The aromatic polyaryl ether having an alkylsulfonic acid and/or an alkali metal salt thereof according to the present invention is not limited in form or shape in its applications. It is applicable in any molded form, such as a membrane, a sheet, a fiber (inclusive of a hollow fiber), and a solid article. The molding method is not limited, either, and includes extrusion, casting, and injection molding. A membrane can be molded by solvent casting, melt casting, and the like. In the case of solvent casting, for instance, the polymer is dissolved in a polar solvent, such as dimethyl sulfoxide, sulfolane, N-methyl-2-pyrrolidone, 1,3-dimethyl-2-imidazolidinone, N,N-dimethylformamide, N,N-dimethylacetamide, methanol, water, and diphenyl sulfone, and the solution is cast on a carrier, followed by evaporation of the polar solvent to provide a membrane.

If desired, the aromatic polyaryl ether of the invention may have part of its sulfonic acid group converted into a metal salt as long as the effects of the invention are not impaired. The aromatic polyaryl ether of the invention may be mixed with a powdered fiber or infiltrated into a fiber, a sheet, a porous membrane, etc. to give a reinforced product. If desired, the aromatic polyaryl ether of the invention may be mixed with an inorganic acid, e.g., phosphoric acid, hypophosphorous acid or sulfuric acid, or a salt thereof, a perfluoroalkylsulfonic acid having 1 to 14 carbon atoms or a salt thereof, a perfluoroalkylcarboxylic acid having 1 to 14 carbon atoms or a salt thereof, an inorganic substance such as platinum, silica gel, silica or zeolite, or any other polymer.

The present invention will now be illustrated in greater detail with reference to Examples. In Examples, various measurements were made as follows.

1) Gas Chromatography-Mass Spectrometry (GC-MS)

Analysis was conducted with GC-MS QP1000 from Shimadzu Corp. using an Ultra Alloy⁺-1 column (film thickness: 0.5 μm; 0.5 mm (i.d.)×15 m) under the following conditions. Column temperature: 70° to 320° C.; temperature rise: 10° C./min; carrier gas: He, 25 ml/min; ionization: E.I.

2) Ion Conductivity

A 5 mm wide film was sandwiched between a Teflon plate and another Teflon plate having platinum wires attached thereto at a 2 mm distance with a slit made between the platinum wires. The ionic conductivity of the film was determined by complex impedance measurement with 3532 LCR Hi-Tester supplied by Hioki E.E. Corp. in a thermostat set at 50° C. and 90% RH.

### EXAMPLE 1

### Synthesis of 2,2-bis(4-hydroxyphenyl)-5-chloropentane

In a flask equipped with a condenser and a tube for introducing nitrogen were put 24.11 g (0.2 mol) of 5-chloro-2-pentanone, 118.2 g (2 mol) of phenol, and 6.25 g (0.04 mol) of 3-mercaptopropanesulfonic acid. The mixture was stirred using a magnetic stirrer in a nitrogen stream at 38° to 42° C. for 18 hours. After the reaction, the resulting solution was washed twice with a large quantity of distilled water. The organic layer was washed successively with 1 liter of a 2 wt % aqueous solution of sodium carbonate and two portions of distilled water, followed by drying at room temperature under reduced pressure. The resultant solid was extracted with ethyl acetate and dried at room temperature under reduced pressure. The resulting solid weighing 12 g was purified by column chromatography on Wako Gel C-200 (from Wako Pure Chemical Industries, Ltd.) using chloroform as a mobile phase, dried at room temperature under reduced pressure to give transparent solid. The H-NMR spectrum (solvent: chloroform; internal standard: TMS) of the product revealed signals at 1.5 to 1.6 ppm (for a methyl group and a methylene group located between methylene groups), 2.1 to 2.2 ppm (for a methylene group next to a methine group), 3.4 to 3.5 ppm (for a methylene group next to a chlorine atom), and 6.6 to 6.8 ppm and 7.0 to 7.1 ppm (each for a phenyl group). The integrated intensity ratio of the signals agreed with that of 2,2-bis(4-hydroxyphenyl)-5-chloropentane. The GC-MS showed a single peak having a molecular weight of 290. These results prove the product to be 2,2-bis(4-hydroxyphenyl)-5-chloropentane with a purity of 100% (by GC analysis).

### Synthesis of sodium 2,2-bis(4-hydroxyphenyl)pentanesulfonate

In a flask equipped with a condenser were put 7.27 g (0.025 mol) of 2,2-bis(4-hydroxyphenyl)-5-chloropentane, 4.73 g (0.038 mol) of sodium sulfite, and 100 g of distilled water. The mixture was refluxed for 18 hours while stirring with a magnetic stirrer. The resulting aqueous solution was filtered to remove the produced viscous matter. The filtrate was washed with ethyl acetate, and the aqueous layer was dried under reduced pressure. The resulting solid was dissolved in butanol, and any insoluble matter was removed by filtration. The filtrate was extracted with distilled water. The aqueous layer was washed with ethyl acetate and dried under reduced pressure. The resultant solid was dissolved in isopropyl alcohol. A large quantity of ethyl acetate was poured in the solution to precipitate a white solid (7.1 g, 80%). The H-NMR spectrum of the solid (solvent: heavy water, internal standard: TMS) revealed signals at 1.4 to 1.7 ppm (for a methyl group and a methylene group located between methylene groups), 2.0 to 2.2 ppm (for a methylene group next to a methine group), 2.7 to 2.9 ppm (a methylene group next to a sulfonic acid group), and 6.8 to 6.9 ppm and 7.0 to 7.1 ppm (each for a phenyl group). The integrated intensity ratio of the signals agreed with that of 2,2-bis(4-hydroxyphenyl)pentanesulfonic acid. The sodium content was found to be 6.63% (theoretical: 6.41%) as a result of the elemental analysis. These results prove the product to be sodium 2,2-bis(4-hydroxyphenyl)pentanesulfonate represented by chemical formula (15):

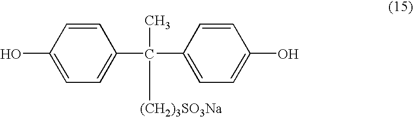

### EXAMPLE 2

### Synthesis of Alkylsulfonated Aromatic Polyether Sulfone

In a flask equipped with a stirrer, a water content meter with a condenser, and a tube for introducing nitrogen were put 3.58 g (0.01 mol) of sodium 2,2-bis(4-hydroxyphenyl)pentanesulfonate obtained in Example 1, 2.54 g (0.01 mol) of bis(4-fluorophenyl) sulfone, 2.07 g (0.015 mol) of potassium carbonate, 30 g of dimethyl sulfoxide, and 20 g of toluene. The mixture was refluxed in a nitrogen stream at 145° to 150° C. for 4 hours. After confirming that release of water came to an end, toluene was removed, and the inner temperature was elevated to 175° C., at which the mixture was maintained for 18 hours. The resulting viscous liquid was filtered to remove solid matter. The filtrate was poured into a large amount of water, and the solid thus precipitated was collected by filtration, dried, and dissolved in N,N-dimethylacetamide in a concentration of 20 wt %. The solution was cast on a glass plate, dried at 150° C., and peeled from the glass plate to obtain a transparent film. The resulting film retained sufficient strength without breakage even on being bent 180°. The film had an ion conductivity of 2.3×10⁻⁴ S/cm.

The film was treated in an 1N aqueous solution of sulfuric acid at 80° C. for 3 hours and washed with water until the washings became neutral. The thus treated film had an ion conductivity of 2.0×10⁻² S/cm and an ion exchange capacity of 1.76 meq/g, which shows that the polymer had been converted to an alkylsulfonic acid by the acid treatment.

### EXAMPLE 3

### Synthesis of Alkylsulfonated Aromatic Polyether Sulfone Copolymer

In a flask equipped with a stirrer, a water content meter with a condenser, and a tube for introducing nitrogen were put 10 g (0.025 mol) of the sodium 2,2-bis(4-hydroxyphenyl)pentanesulfonate obtained in Example 1, 5.79 g (0.025 mol) of 2,2-bis(4-hydroxyphenyl)propane, 12.89 g (0.05 mol) of bis(4-fluorophenyl) sulfone, 9.12 g of potassium carbonate, 150 g of dimethyl sulfoxide, and 50 g of toluene. The mixture was refluxed in a nitrogen stream at 145° to 150° C. for 4 hours. After confirming that release of water came to an end, toluene was removed, and the temperature was raised up to 175° C., at which the reaction mixture was kept for 18 hours. The resulting viscous liquid was filtered to remove any solid matter, and the filtrate was poured into a large quantity of water to precipitate a solid. The solid was collected by filtration, dried, and dissolved in N,N-dimethylacetamide in a concentration of 20 wt %. The polymer solution was cast on a glass plate, dried at 150° C., and peeled from the glass plate to give a transparent film. The resulting film retained sufficient strength without breakage even on being bent 180°.

The film was treated in an 1N aqueous solution of sulfuric acid at 80° C. for 3 hours and washed with water until the washings became neutral. The thus treated film had an ion conductivity of 2.6×10⁻³ S/cm. The ion exchange capacity of the film was 0.92 meq/g, which shows that the polymer had been converted to an alkylsulfonic acid by the acid treatment.

### EXAMPLE 4

### Synthesis of 2,2-bis(4-hydroxyphenyl)-6-chlorohexane

In a flask equipped with a condenser and a tube for introducing nitrogen were put 26.92 g (0.2 mol) of 6-chloro-2-hexanone, 118.2 g (2 mol) of phenol, and 6.25 g (0.04 mol) of 3-mercaptopropanesulfonic acid. The mixture was stirred with a magnetic stirrer in a nitrogen stream at 40° C. for 18 hours. After the reaction, the resulting solution was washed twice with a large quantity of distilled water. The organic layer was washed successively with 1 liter of a 2 wt % aqueous solution of sodium carbonate and two portions of distilled water, followed by drying at room temperature under reduced pressure. The resulting viscous matter was extracted with ethyl acetate and dried at room temperature under reduced pressure. The resulting solid weighing 20 g was purified by column chromatography on Wako Gel C-200 (from Wako Pure Chemical Industries, Ltd.) using chloroform as a mobile phase, dried at room temperature under reduced pressure to give transparent viscous matter. The H-NMR spectrum (solvent: dimethyl sulfoxide; internal standard: TMS) of the product revealed signals at 1.1 to 1.2 ppm (for a second methylene group from a methine group), 1.4 to 1.5 ppm (for a methyl group next to a methine group), 1.5 to 1.6 ppm (for a methylene group next to a methine group), 2.4 to 2.5 ppm (for a methylene group next to a chlorine atom), 1.9 to 2.0 ppm (for a second methylene group from a chlorine atom), and 6.6 to 6.7 ppm and 6.9 to 7.0 ppm (each for a phenyl group). The integrated intensity ratio of the signals agreed with that of 2,2-bis(4-hydroxyphenyl)-6-chlorohexane. The GC-MS showed a single peak having a molecular weight of 304. These results prove the product to be 2,2-bis(4-hydroxyphenyl)-6-chlorohexane with a purity of 100% (by GC analysis).

### Synthesis of sodium 2,2-bis(4-hydroxyphenyl)hexanesulfonate

In a flask equipped with a condenser were put 7.62 g (0.025 mol) of 2,2-bis(4-hydroxyphenyl)-6-chlorohexane, 4.73 g (0.038 mol) of sodium sulfite, and 100 g of distilled water. The mixture was refluxed for 20 hours while stirring with a magnetic stirrer. The resulting aqueous solution was filtered to remove the produced viscous matter. The filtrate was washed with ethyl acetate, and the aqueous layer was dried under reduced pressure. The resulting solid was dissolved in butanol, and any insoluble matter was removed by filtration. The filtrate was extracted with distilled water. The aqueous layer was washed with ethyl acetate and dried under reduced pressure. The resultant solid was dissolved in isopropyl alcohol. A large quantity of ethyl acetate was poured in the solution to precipitate a white solid (7.9 g, 85%). The H-NMR spectrum of the solid (solvent: dimethyl sulfoxide, internal standard: TMS) revealed signals at 1.4 to 1.6 ppm (for a methyl group next to a methine group), 1.4 to 1.6 ppm (for a methylene group next to a methine group), 2.4 to 2.6 ppm (a methylene group next to a sulfonic acid group), 1.9 to 2.1 ppm (a second methylene group from a sulfonic acid group), and 6.6 to 6.7 ppm and 6.9 to 7.0 ppm (each for a phenyl group). The integrated intensity ratio of the signals agreed with that of sodium 2,2-bis(4-hydroxyphenyl)hexanesulfonate. The sodium content was found to be 6.24% (theoretical: 6.17%) as a result of the elemental analysis. These results prove the product to be sodium 2,2-bis(4-hydroxyphenyl)hexanesulfonate represented by chemical formula (16):

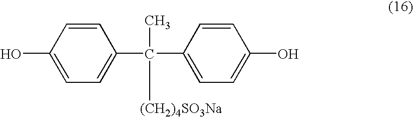

### EXAMPLE 5

### Synthesis of alkylsulfonated aromatic polyether Sulfone

In a flask equipped with a stirrer, a water content meter with a condenser, and a tube for introducing nitrogen were put 3.72 g (0.01 mol) of sodium 2,2-bis(4-hydroxyphenyl)hexanesulfonate obtained in Example 4, 2.54 g (0.01 mol) of bis(4-fluorophenyl) sulfone, 2.07 g (0.015 mol) of potassium carbonate, 30 g of dimethyl sulfoxide, and 20 g of toluene. The mixture was refluxed in a nitrogen stream at 145° to 150° C. for 4 hours. After confirming that release of water came to an end, toluene was removed, and the inner temperature was elevated to 175° C., at which the mixture was maintained for 18 hours. The resulting viscous liquid was filtered to remove solid matter. The filtrate was poured into a large amount of water, and the solid thus precipitated was collected by filtration, dried, and dissolved in dimethyl sulfoxide in a concentration of 20 wt %. The solution was cast on a glass plate, dried at 200° C., and peeled from the glass plate to obtain a transparent film. The resulting film retained sufficient strength without breakage even on being bent 180°. The film had an ion conductivity of 3.1×10⁻⁴ S/cm.

The film was treated in an 1N aqueous solution of sulfuric acid at 80° C. for 3 hours and washed with water until the washings became neutral. The thus treated film had an ion conductivity of 1.8×10⁻² S/cm and an ion exchange capacity of 1.51 meq/g. This indicates that the polymer had been converted to an alkylsulfonic acid by the acid treatment.

### EXAMPLE 6

### Synthesis of 1,7-dichloro-4-heptanone

In a flask equipped with a condenser and a tube for introducing nitrogen were put 172.18 g (2 mol) of γ-butyrolactone and 192.9 g of a 28% methanol solution of sodium methoxide (2 mol) and refluxed in a nitrogen stream at 120° C. for 3 hours while stirring by a magnetic stirrer. After the reaction, methanol was removed from the resulting solution, and 500 ml of 12N concentrated sulfuric acid was added thereto. The mixture was refluxed in a nitrogen stream at 120° C. for 15 minutes while stirring with a magnetic stirrer. The resulting brown oily liquid was extracted with diethyl ether, neutralized with 500 ml of a 20 wt % aqueous solution of potassium carbonate, and distilled at 110° C. under reduced pressure (0.05 mmHg) to give a clear liquid. The H-NMR spectrum (solvent: chloroform; internal standard: TMS) of the liquid revealed signals at 1.9 to 2.0 ppm (for a second methylene group from a chlorine atom), 2.6 to 2.7 ppm (for a methylene group next to a carbonyl group), and 3.6 to 3.7 ppm (for a methylene group next to a chlorine atom). The integrated intensity ratio of the signals agreed with that of 1,7-dichloro-4-heptanone. The GC-MS showed a single peak having a molecular weight of 182. These results prove the product to be 1,7-dichloro-4-heptanone with a purity of 100% (by GC analysis).

### Synthesis of 2,2-bis(4-hydroxyphenyl)-1,7-dichloroheptane

In a flask equipped with a condenser and a tube for introducing nitrogen were put 73.23 g (0.4 mol) of 1,7-dichloro-4-heptanone, 376.4 g (4 mol) of phenol, and 12.5 g (0.08 mol) of 3-mercaptopropanesulfonic acid. The mixture was stirred in a nitrogen stream at 40° C. for 48 hours with a magnetic stirrer. After the reaction, the resulting solution was washed three times with a large quantity of distilled water. The organic layer was washed with 1 liter of a 2 wt % aqueous solution of sodium carbonate and two portions of distilled water, followed by drying at room temperature under reduced pressure. The resulting viscous matter was extracted with ethyl acetate and dried at room temperature under reduced pressure. The resulting viscous liquid weighing 20 g was purified by column chromatography on Wako Gel C-300 (from Wako Pure Chemical Industries, Ltd.) using chloroform and a 10 wt % solution of acetone in chloroform as a mobile phase, and dried at room temperature under reduced pressure to give a transparent viscous substance. The H-NMR spectrum (solvent: chloroform; internal standard: TMS) of the viscous substance revealed signals at 1.0 to 1.2 ppm (for a second methylene group from a methine group), 1.9 to 2.3 ppm (for a methylene group next to a methine group), 3.3 to 3.5 ppm (for a methylene group next to a chlorine atom), and 6.6 to 6.7 ppm and 6.9 to 7.0 ppm (each for a phenyl group). The integrated intensity ratio of the signals agreed with that of 2,2-bis(4-hydroxyphenyl)-1,7-dichloroheptane. The GC-MS showed a single peak having a molecular weight of 352. These results prove the product to be 2,2-bis(4-hydroxyphenyl)-1,7-dichloroheptane with a purity of 100% (by GC analysis).

### Synthesis of sodium 2,2-bis(4-hydroxyphenyl)-1,7-heptanedisulfonate

In a flask equipped with a condenser were put 8.83 g (0.025 mol) of 2,2-bis(4-hydroxyphenyl)-1,7-dichloroheptane, 9.46 g (0.076 mol) of sodium sulfite, and 150 g of distilled water. The mixture was refluxed for 20 hours while stirring using a magnetic stirrer. The resulting aqueous solution was filtered to remove the produced viscous matter. The filtrate was washed with ethyl acetate, and the aqueous layer was dried under reduced pressure. The resulting solid was dissolved in ethanol, and any insoluble matter was removed by filtration. Ethanol was removed from the filtrate to give a white solid, which was washed with ethyl acetate and extracted with distilled water. The aqueous layer was washed with hexane and dried under reduced pressure. The resulting solid was dissolved in isopropyl alcohol, and a large quantity of ethyl acetate was poured in the solution to precipitate a white solid (1.83 g, 15%). The H-NMR spectrum of the resulting white solid (solvent: dimethyl sulfoxide, internal standard: TMS) revealed signals at 1.0 to 1.2 ppm (for a second methylene group from a methine group), 1.9 to 2.3 ppm (for a methylene group next to a methine group), 3.3 to 3.5 ppm (for a methylene group next to a chlorine atom), and 6.6 to 6.7 ppm and 6.9 to 7.0 ppm (each for a phenyl group). The integrated intensity ratio of the signals agreed with that of sodium 2,2-bis(4-hydroxyphenyl)-1,7-heptanedisulfonate. The sodium content was found to be 9.78% (theoretical: 9.41%) as a result of the elemental analysis. These results prove the product to be sodium 2,2-bis(4-hydroxyphenyl)-1,7-heptanedisulfonate represented by chemical formula (17):

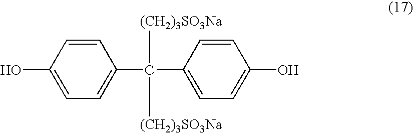

### EXAMPLE 7

### Synthesis of Alkylsulfonated Aromatic Polyether Sulfone Copolymer

In a flask equipped with a stirrer, a water content meter with a condenser, and a tube for introducing nitrogen were put 12.21 g (0.025 mol) of the sodium 2,2-bis(4-hydroxyphenyl)-1,7-heptanedisulfonate obtained in Example 6, 5.79 g (0.025 mol) of 2,2-bis(4-hydroxyphenyl)propane, 12.89 g (0.05 mol) of bis(4-fluorophenyl) sulfone, 9.12 g of potassium carbonate, 150 g of dimethyl sulfoxide, and 50 g of toluene. The mixture was refluxed in a nitrogen stream at 145° to 150° C. for 4 hours. After confirming that release of water came to an end, toluene was removed, and the temperature was raised up to 175° C., at which the reaction mixture was kept for 18 hours. The resulting viscous liquid was filtered to remove any solid matter, and the filtrate was poured into a large quantity of water to precipitate a solid. The solid was collected by filtration, dried, and dissolved in dimethyl sulfoxide in a concentration of 20 wt %. The polymer solution was cast on a glass plate, dried at 160° C., and peeled from the glass plate to give a transparent film. The resulting film retained sufficient strength without breakage even on being bent 180°.

The film was treated in an 1N aqueous solution of sulfuric acid at 80° C. for 3 hours and washed with water until the washings became neutral. The thus treated film had an ion conductivity of 3.5×10⁻² S/cm. The ion exchange capacity of the film was 1.62 meq/g, which shows that the polymer had been converted to an alkylsulfonic acid by the acid treatment.

### INDUSTRIAL APPLICABILITY

The present invention provides a novel bisphenol compound having an alkylsulfonic acid and/or an alkali metal salt thereof which is useful as a starting material of polyelectrolytes comprising an aromatic polyether sulfone, an aromatic polyether ketone, an aromatic polyester, a polycarbonate, an epoxy resin, a phenol resin, etc.

The present invention also provides a novel aromatic polyaryl ether having an alkylsulfonic acid and/or an alkali metal salt thereof in its side chain, such as an aromatic polyether sulfone or an aromatic polyether ketone, which is useful as a molding material of a polyelectrolyte or a polyelectrolyte membrane used in fuel cells, secondary batteries, capacitors, ion exchange resins, ion exchange membranes, separation membranes, and so forth.

1. A bisphenol compound represented by chemical formula (1): 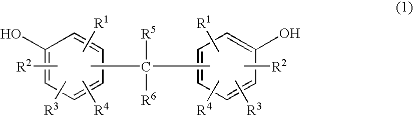
wherein R¹, R², R³, and R⁴ each independently represent a hydrogen atom or an alkyl group having 1 to 3 carbon atoms; and R⁵ and R⁶ each independently represent a hydrogen atom, an alkyl group having 1 to 6 carbon atoms, an aromatic group or a structure represented by chemical formula (2):
—R⁷ (CH₂)ₙSO₃X (2)
wherein R⁷ represents nothing or an aromatic group; X represents a hydrogen atom or an alkali metal; and n represents an integer of from 1 to 12,
provided that at least one of R⁵ and R⁶ represents the structure of chemical formula (2).

2. The bisphenol compound according to claim 1, wherein the chemical formula (1) is chemical formula (3): 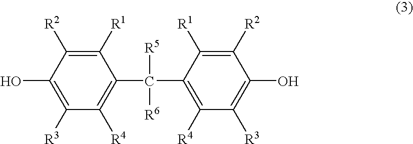
wherein R¹, R², R³, R⁴, R⁵, and R⁶ are as defined in chemical formula (1).

3. An aromatic polyaryl ether characterized by having a structural unit represented by chemical formula (4): 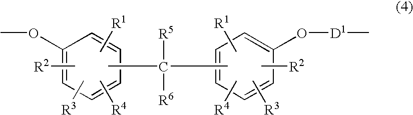
wherein R¹, R², R³, and R⁴ each independently represent a hydrogen atom or an alkyl group having 1 to 3 carbon atoms; R⁵ and R⁶ each independently represent a hydrogen atom, an alkyl group having 1 to 6 carbon atoms, an aromatic group or a structure represented by chemical formula (5):
—R⁷ (CH₂)ₙSO₃X (5)
wherein R⁷ represents nothing or an aromatic group; X represents a hydrogen atom or an alkali metal; and n represents an integer of from 1 to 12,
provided that at least one of R⁵ and R⁶ represents the structure of chemical formula (5); and D¹ represents a structure represented by chemical formula (6): 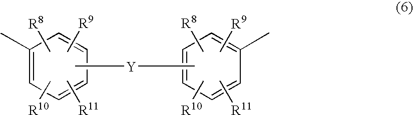
wherein R⁸, R⁹, R¹⁰, and R¹¹ each independently represent a hydrogen atom, a halogen atom, an alkyl group having 1 to 3 carbon atoms or a nitro group; and Y represents —S(═O)₂ or C(═O)—,
or chemical formula (7): 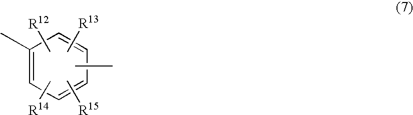
wherein R¹², R¹³, R¹⁴, and R¹⁵ each independently represent a hydrogen atom, a halogen atom, an alkyl group having 1 to 3 carbon atoms, a nitro group or a cyano group, provided that at least one of them is a nitro group or a cyano group.

4. The aromatic polyaryl ether according to claim 3, wherein the structural unit represented by chemical formula (4) is a structural unit represented by chemical formula (8): 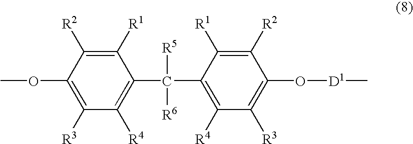
wherein R¹, R², R³, R⁴, R⁵, R⁶, and D¹ are as defined in chemical formula (4).

5. An ion conductive polymer comprising the aromatic polyaryl ether according to claim 3.

6. A polyelectrolyte membrane comprising the aromatic polyaryl ether according to claim 3.

7. A fuel cell having the polyelectrolyte membrane according to claim 6.

8. An ion conductive polymer comprising the aromatic polyaryl ether according to claim 4.

9. A polyelectrolyte membrane comprising the aromatic polyaryl ether according to claim 4.
